(19) United States
(12) Patent Application Publication
(10) Pub. No.: US 2004/0049283 A1
(43) Pub. Date: Mar. 11, 2004

(54) Medical implant and method of reducing back pain

(76) Inventor: Tushar Patel, McLean, VA (US)

Correspondence Address: GREENBERG-TRAURIG, 1750 TYSONS BOULEVARD, 12TH FLOOR, MCLEAN, VA, 22102, US

(21) Appl. No.: 10/452,186

(22) Filed: Jun. 3, 2003

Related U.S. Application Data
(60) Provisional application No. 60/385,745, filed on Jun. 4, 2002.

(51) Int. Cl. A61F 2/44

(52) U.S. Cl. 623/17.16; 623/17.11; 623/901

(57) ABSTRACT

An expandable medical implant capable of being inserted between adjacent vertebral endplates in a spine. The implant, when in an expanded position, includes a thicker peripheral portion and a thinner interior portion. Also, a method of manufacturing an expandable medical implant having a thicker peripheral portion and a thinner interior portion. Further, a method for reducing back pain that includes placing a medical implant between two adjacent vertebral endplates and positioning the implant such that the implant distributes stresses toward stronger portions of the endplates when pressure is placed on the implant.

### CROSS-REFERENCE TO RELATED APPLICATIONS

The present application claims priority from U.S. Provisional Patent Application No. 60/385,745 (hereafter, “the '745 Application”), which was filed on Jun. 4, 2002 entitled “Nucleus Implant that Prevents Endplate Subsidence”. The '745 Application is hereby incorporated herein in its entirety by reference.

### BACKGROUND OF THE INVENTION

1. Field of the Invention

The field of the present invention includes medical implants, methods of manufacturing the same and methods of reducing back pain.

2. Description of the Related Art:

A healthy human spine includes a set of vertebrae and a set of discs that are positioned between the vertebrae. Each disc includes two endplates, a gelatinous and relatively soft nucleus, and a fibrous and relatively rigid annulus. The nucleus is located in the center of the disc, the annulus surrounds the nucleus, one of the endplates is positioned above the nucleus and annulus, and the other endplate is positioned below the nucleus and annulus. The endplates are adjacent to vertebral bodies on adjacent vertebrae and connect the disc to the vertebral bodies.

Surgical implants (i.e., prostheses) have been commonly used to remedy a variety of ailments in the spine and in other human joints. For example, surgical implants have been used to replace hips and knee joints and to treat intervertebral disc abnormalities.

Some surgical implants, in the form of arthroscopically implantable prostheses, have been designed to restore function to a diseased joint, as disclosed in U.S. Pat. No. 5,344,459 to Swartz (hereafter Swartz '459). The prostheses disclosed in Swartz '459 are inflatable and designed to be implanted into weight-bearing joints such as human knees. The Swartz '459 prostheses include either one or two rings that are sized and shaped to fit within the knee joint. Each of these rings may include separate compartments for stabilization and support.

The Swartz '459 prostheses were designed to be inflated arthroscopically and to be easily implanted with and disconnected from an arthroscope. However, they were designed primarily to provide pain relief, support, mobility, and correction of joint angulation for the human knee. Hence, they were not well suited to meet the peculiar requirements of the human spine.

For example, if a Swartz '459 prostheses were implanted between the endplates of a spinal disc and if a patient were to put pressure on the spine by standing or sitting up, the ring structure would distribute a significant amount of the pressure to the interior portions of the endplates. Accordingly, there is a likelihood that the prosthesis would lead to further spinal damage upon loading. In addition, a Swartz '459 prosthesis with its near circular configuration would have an undesirable amount of mobility within the spine since this type of prosthesis is not well suited for the unusual structures and more complex geometry of the spine.

Other examples of surgical implants include the joint endoprostheses disclosed in U.S. Pat. No. 4,932,969 to Frey et al. (hereafter Frey '969). The Frey '969 endoprostheses were designed to be used intervertebrally and may include a hollow body in the form of a toroidal ring. This ring defines an annular cavity that contains an incompressible fluid medium. Unlike the Swartz '459 prostheses, however, the Frey '969 prostheses were not designed to be inflatable. Therefore, insertion and implantation of the Frey '969 prostheses necessitates that a relatively large path be surgically created through the spine to allow for proper placement between adjacent endplates. Unpredictable stability due to variations in healing with more invasive surgical techniques lends some uncertainty to the long term stability of such devices. In addition, as with the Swartz '459 prostheses, if pressure were applied, the Frey '969 prostheses would distribute a significant amount of the pressure to the relatively weak interior portions of the endplates and would therefore increase the likelihood of further end plate degradation.

Hence, both in the context of intervertebral implants, and in other environments where selective distribution of pressure among corporeal structures would lend stability and minimize degradation, there is a need for expandable medical implants that have only a limited amount of mobility within a joint. There is also a need for medical implants that, when implanted between the vertebral body endplates of a disc or similar structure, protect the weaker interior portions of the endplates by distributing pressure and/or stress away from those weaker portions and toward the stronger portions, which in the case of the spine is amidst the outer position of the endplates. Further, there is a need for methods of manufacturing such medical implants. Still further, there is a need for improved methods of reducing back pain, typically through the use of such improved medical implants.

### SUMMARY OF CERTAIN EMBODIMENTS OF THE INVENTION

Accordingly, certain embodiments of the present invention are directed at expandable medical implants (i.e., prostheses) that substantially obviate one or more of the problems due to the limitations and disadvantages of the related art. Certain embodiments of the present invention are also directed at methods of manufacturing medical implants and at methods of reducing back pain that obviate one or more of the limitations and disadvantages of the related art.

According to one embodiment of the present invention, a medical implant is provided that includes an expandable cover or shell having an interior portion and a peripheral portion that surrounds the interior portion. The implant is manufactured to have appropriate dimensions for insertion thereof between a first vertebral endplate and a second vertebral endplate. When in an expanded position, the peripheral portion of the implant has a thicker cross-section than the interior portion of the implant.

According to another embodiment of the present invention, a method of manufacturing a medical implant is provided. The method includes forming an expandable cover or shell that itself includes an interior portion and a peripheral portion that surrounds the interior portion. The method also includes forming the implant to have appropriate dimensions for insertion between a first vertebral endplate and a second vertebral endplate. The method further includes forming an implant that, when in an expanded position, has a peripheral portion that is thicker than the interior portion of the implant.

According to yet another embodiment of the present invention, a method of reducing back pain is provided by deploying the described implant(s) between vertebral bodies. The method includes placing a medical implant between two adjacent vertebral endplates. The method also includes positioning the implant such that, pursuant to application of pressure and/or a force to the implant, stress resulting from the pressure and/or force is preferentially distributed toward stronger portions of the vertebral endplates and away from weaker portions of the vertebral endplates such as the interior.

According to another embodiment of the present invention, an intervertebral disc implant is provided. This implant includes a biocompatible, pre-shaped expandable member that has been adapted to fit between two adjacent vertebral bodies. The member has a bottom surface for placement against an uncus and the member also has a top surface for placing against an inferior endplate of an adjacent vertebra. The implant contains at least one concavity on one of its surfaces.

According to yet another embodiment of the present invention, a method of reducing back pain is provided. The method includes placing a medical implant between two adjacent vertebral endplates. The method also includes positioning the implant such that, pursuant to application of a force thereto, stress resulting from the force is distributed between the endplates in a normalized manner.

The principals of the present invention can be applied to other joints and settings where preferential pressure loading to accommodate natural injury-induced characteristics of hard and soft tissue would be advantageous.

Features and advantages of the present invention are set forth in the description which follows, and in part will be apparent from the description, or may be learned by practice of the invention. The objectives and other advantages of the invention will be realized and attained by the structures and methods particularly pointed out in the written description and claims hereof as well as the appended drawings. It is to be understood that both the foregoing general description and the following detailed description are exemplary and explanatory and are intended to provide further explanation of the invention as claimed. They are not intended to limit the potential applications of the invention to only those described. Likewise, the drawings depict several potential embodiments of accomplishing the present invention and are not intended as limitations on other potential embodiments.

### BRIEF DESCRIPTION OF THE FIGURES

The accompanying figures, which are incorporated into and constitute part of this specification, illustrate several embodiments of the present invention. Together with the detailed description below, these figures serve to explain the invention in greater detail.

In the figures:

FIG. 1 illustrates a top view of a medical implant according to certain embodiments of the present invention;

FIGS. 2A-2E are cross-sectional views of various representative medical implants where covers of the implants are in expanded positions;

FIG. 3A illustrates a perspective view of an embodiment of the present invention wherein the cover includes several separate compartments formed therewithin; and

FIG. 3B provides a perspective view of several compartments of a medical implant that have been formed separately and subsequently joined to form a portion of a cover of a medical implant.

### DETAILED DESCRIPTION OF CERTAIN EMBODIMENTS

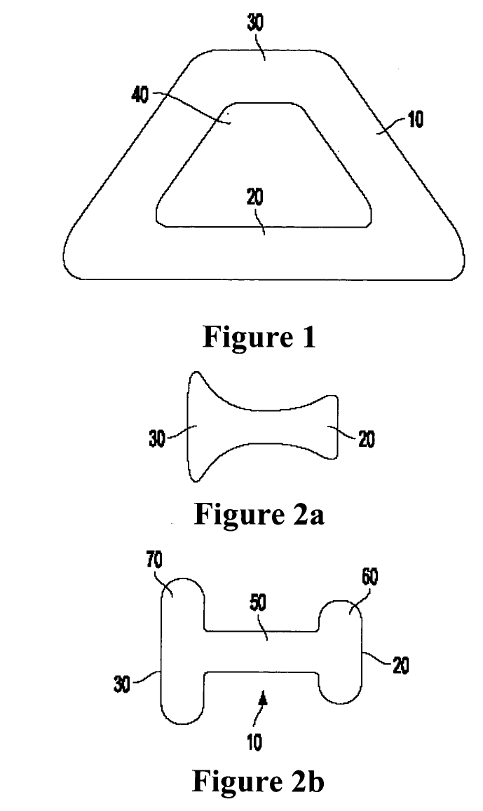

FIG. 1 illustrates a top view of a medical implant according to certain embodiments of the present invention. The medical implant in FIG. 1 includes an expandable cover 10 that has an interior portion 20 which surrounds a void 40. The cover 10 also has a peripheral portion 30 that surrounds the interior portion 20.

Though medical implants according to the present invention may be used in a variety of locations in a patient's body (e.g., in any of the joints in a patient's body), the cover 10, according to certain embodiments, is appropriately dimensioned to fit between two vertebrae of the human spine or, more specifically, between two vertebral body endplates. No particular restrictions are made on where the interior portion 20 of the cover 10 ends and where the peripheral portion 30 begins. However, according to certain preferred embodiments, after a vertebral implant that has been inserted in a patient's body, the exterior portion 30 is substantially adjacent to portions of the endplates that are more structurally sound (i.e., more rigid, stronger, and/or that have the capacity to support a larger load) while the interior portion 20 is substantially adjacent to portions of the endplate that are less structurally sound (i.e., less rigid, weaker, and/or that have a lesser capacity for supporting a load). According to certain other embodiments, after insertion into a patient's body, the exterior portion 30 is substantially adjacent to the annulus of the vertebral disc and the interior portion 20 is substantially adjacent to the nucleus of the same disc. According to yet other embodiments, the interior portion 20 extends over approximately half of the distance from the center of the implant (or, if present, the edge of the void 40) to the exterior edge of the implant. The exterior portion 30 then extends over the remaining half of the distance.

The void 40 illustrated in FIG. 1 is optional and therefore not present in all medical implants according to the present invention. When the void 40 is present, it is typically positioned adjacent to at least a portion of the nucleus portion of the disc. According to certain embodiments, the cover 10 is sized to fit in the annulus portion of the disc, thereby reducing the likelihood that the cover 10 will slip or move once between the endplates.

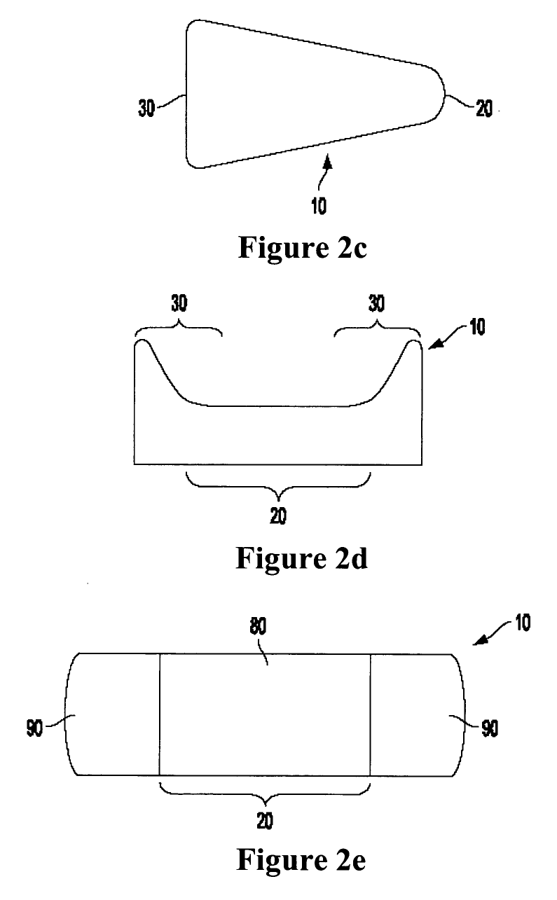

The cover 10 may be inserted in a patient's body either in a collapsed or expanded state or position. When the cover 10 is properly positioned between two vertebral body endplate in an expanded position, the cover 10 may either extend from one endplate to the other or, in the alternative, may extend over only a portion of that distance. FIGS. 2A-2E are cross-sectional views of various representative medical implants where the covers 10 are expanded.

In FIGS. 2A-2C, the left-most portions of the figures illustrate peripheral portions 30 of covers 10 that include voids 40. The right-most portions of the figures illustrate interior portions 20 of covers 10 that include voids 40. In FIGS. 2D and 2E, the left-most and right-most portions of the figures illustrate peripheral portions 30 of covers 10 that do not include a void 40 and the center portions of the figures illustrate interior portions 20 of covers 10 that do not include voids 40.

FIG. 2A illustrates the cross-section of a cover 10 having a bi-concave geometry where the center of the cross-section is thinner than either end of the cross-section. This bi-concave cross-sectional geometry may be designed such that the thickness of the cross-section in the peripheral portion 30 of the cover 10 is greater than the thickness of the cross-section in the interior portion 20. In such a design, when the implant cover 10 forms a closed loop and is positioned between and compressed by two vertebral body endplates, stress is distributed to the stronger, outer portions of the endplates and away from the relatively weak inner portions of the endplates (e.g., portions of the endplates adjacent to the nucleus of the disc). According to other embodiments, the cover may be designed to distribute stress to those areas of the patient's body that are least likely to be adversely affected by the stress.

In FIG. 2B, the cover 10 has a substantially I-shaped cross-section with a horizontal section 50 that is positioned between two vertical sections 60, 70. In the cover 10 illustrated in FIG. 2B, the vertical section 60 that is situated in the interior portion 20 has a shorter length than the vertical section 70 that is situated in the peripheral portion 30. This type of cross-sectional geometry, when included in an implant that forms a closed loop and that is positioned between and compressed by two vertebral body endplates, distributes stress to the stronger portions of the endplates and away from the relatively weak inner portions thereof.

In FIG. 2C, the peripheral portion 30 of the cover 10 is substantially thicker than the interior portion 20 and a gradual tapering of the thickness is illustrated between the two portions 20, 30. However, covers 10 with less gradual tapering are also within the scope of the present invention, as are covers with step-like (i.e., discontinuous or abrupt) decreases in thickness. Regardless of whether the tapering is gradual or abrupt, this type of cross-sectional geometry distributes stress to the stronger portions of the endplates in a closed-loop-type implant such as those discussed above.

FIG. 2D illustrates the cross-sectional geometry of a cover with a concave top and a flat bottom. In FIG. 2D, the cover 10 is substantially thicker in its peripheral portion 30 than in its interior portion 20. Therefore, when the cover 10 is positioned between and compressed by two endplates, stress is distributed preferentially to the outer, stronger portions of the endplates.

The cover 10 shown in FIG. 2E includes a compliant material 80 (e.g., a sponge-like material) in the interior portion 20 thereof and a load-bearing material 90 in the peripheral portion 30 thereof. The thickness of the cover 10 illustrated in FIG. 2E is substantially constant across the entire cross-section shown, but other cross-sectional geometries that include a compliant material and a load-bearing material are also within the scope of the present invention. When a cover 10 such as the one illustrated in FIG. 2E is subjected to a load, the peripheral portions 30 of the cover 10 support the load and the interior portion remains relatively stress-free. Hence, stress resulting from the load is preferentially distributed to the stronger portions of the endplates when the implant is positioned in a patient's body.

The cover 10 may include (or, in some cases, may be made entirely from) materials such as, but not limited to, hydrophilic materials, bioresorbable materials, inert materials, fluids whose hydrostatic properties may be controlled from a distance (e.g., fluids whose viscosities may be controlled by magnetic fields), and/or biocompatible materials. When a hydrophilic material (e.g., a polyvinyl alcohol) is included, the material typically absorbs liquid (e.g., water or other fluids present in a patient's body) after being positioned in a patient's body (e.g., in a joint or between two vertebrae). Upon absorption of the liquid, the cover 10 typically expands. When a bioresorbable material (e.g., polydioxanone) is included in the cover 10, the material typically gets gradually incorporated into the patient's body over time. This incorporation prevents movement of the cover 10 over time and adds structural stability to the patient's spine. When an inert material (e.g., expanded poly-tetrafluoroethylene) is used, no adverse interactions with the patient's body are expected. This typically reduces the chances that the implant will be rejected. Biocompatible materials (e.g., polyvinyl alcohols, polydioxanone, expanded poly-tetrafluoroethylene, silicon, etc.) also have the advantage that they are less susceptible to rejection by the patient's body.

According to certain embodiments, a filler is included in the medical implant. When included, the filler is typically positioned within the expandable cover 10 and typically includes materials such as, but not limited to, hydrogels, alcohols (e.g., polyvinyl alcohols), inert gases, inert fluids, fluids whose hydrostatic properties may be controlled from a distance, and biocompatible materials. A hydrogel may be inserted into the cover 10 either in a fully-hydrated state or in a non- or partially-hydrated state. If the hydrogel is not fully hydrated before being inserted into the cover 10, the hydrogel, once in position, may be hydrated and expanded via exposure to fluids in a patient's body (i.e., as fluids travel through the cover 10). Depending upon the permiability of the cover material selected, selective hydration may also be used to selectively adjust dimensions and pressure.

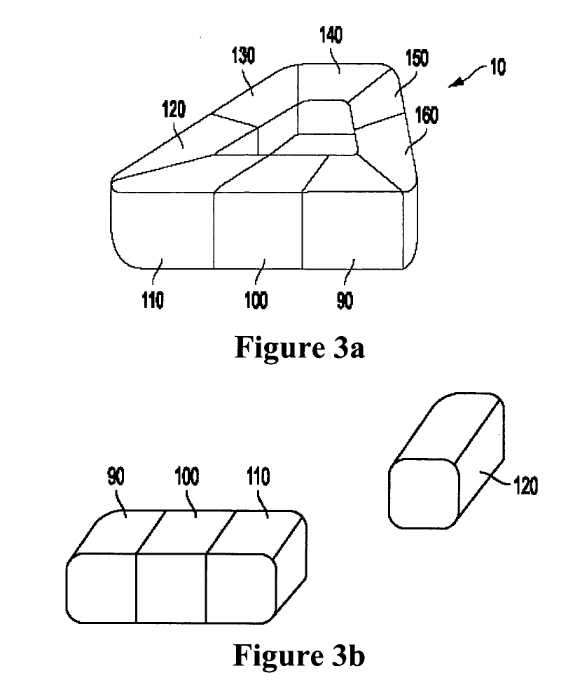

FIG. 3A illustrates a perspective view of an embodiment of the present invention wherein the cover 10 includes several separate compartments 90, 100, 110, 120, 130, 140, 150, 160 formed therewithin. According to certain embodiments, the compartments can be separately expanded and each contain a filler liquid, solid, or gas. If the compartments are filled with either a fluid or a gas, the fluid or gas is not capable of flowing from one compartment to another in certain embodiments of the present invention but is able to flow or permeate through compartment walls according to certain other embodiments. The net result is more localized pressurization which may advantageously reduce any tendency toward migration.

FIG. 3B provides a perspective view of several compartments 90, 100, 110, 120 of a medical implant that have been formed separately and subsequently joined to form a portion of a cover 10. When separately-formed compartments are used, each of these compartments may be inserted into a patient's body individually (or in small groups) and joined once inside the body. In order to join separately formed compartments, adhesives, locking means, and/or other joining methods know to those skilled in the art may be used. Regardless of whether the compartments described above are formed as separate entities or are formed within a cover 10 after the cover 10 has been formed, no particular limit exists on the number of compartments that may be included in a medical implant.

According to certain embodiments of the present invention, the medical implant includes a cover that has one or more textured surfaces. According to these embodiments, one or more surfaces of the cover may be microtextured and/or macrotextured. For example, one or more of the surfaces of the cover that contact a vertebral endplate may be roughened, may include ridges, or may be otherwise patterned to promote and/or enhance adhesion, lubriciousness, susceptibility to biological incorporation into a patient's body, etc.

To reduce the chance that a medical implant will slide or otherwise move once the implant is in a desired position between two vertebral bodies and/or endplates, the cover of the implant may include at least one surface that is anatomically contoured to the bony landmarks found on at least one of the vertebral bodies and/or endplates to which the surface is adjacent. If an anatomically contoured surface is to be included, one or both of the vertebral bodies and/or endplates adjacent to the cover are typically analyzed via Magnetic Resonance Imaging (MRI), X-ray imaging, or some other analytical method in order to determine the topography of the landmarks. The data collected during this analysis is then used to manufacture an implant that is particularly designed for a specific patient's body. If the implant is to be used in a location other than between two vertebral bodies and/or endplates, the topography of rigid portions surrounding the alternate location may also be analyzed and an implant may be designed accordingly.

According to one illustrative and non-limiting embodiment of the present invention, an intervertebral disc implant is provided that includes a biocompatible preshaped expandable member (i.e., a cover, a bladder, etc.). This member is adapted to fit between two adjacent vertebral bodies, preferably between two adjacent endplates. The member includes a bottom surface and, when the member is inserted between vertebrae in the cervical spine, the bottom surface is typically placed against the uncus of a first adjacent vertebra. The member also includes a top surface that is typically placed against an inferior endplate of a second adjacent vertebra. Preferably, the implant contains at least one concavity on either its bottom or top surface.

Medical implants according to the present invention may be manufactured in a number of ways. For example, a medical implant may be manufactured by forming an expandable cover with an interior portion and a peripheral portion that surrounds the interior portion. If the medical implant is to be positioned in a patient's spine, the implant typically is formed to have dimensions that are appropriate for insertion of the implant between two vertebral body endplates. In some cases, the implants have dimensions that allow them to fit in the annulus of a spinal disc. Preferably, the implant is manufactured such that, when in the expanded position, the peripheral portion of the of the cover has a thicker cross-section than the interior portion of the cover.

According to certain embodiments of the above-discussed method, when the implant is designed to be positioned in a patient's spine, the implant is properly sized using precise geometric dimensions of at least one of the vertebral bodies and/or endplates. These dimensions are typically obtained by any of the methods of determining topography described above. If the implant is to be positioned at a location that is not in a patient's spine, the method of manufacturing the implant may include determining and using the precise geometric dimensions of the rigid portions adjacent to where the implant is to be positioned.

When positioned between two vertebral bodies and/or endplates, medical implants such as, but not limited to, those described above may be used to reduce back pain in a patient. Implants are particularly useful in relieving pain when a patient has suffered the degeneration of a disc. However, implants may be used in a variety of other situations.

According to certain embodiments of the present invention, methods of reducing back pain include placing one or more expandable medical implant between two or more adjacent vertebral bodies and/or endplates while each implant is in a collapsed state (i.e., while the implant is relatively small and/or flat). The implants may be positioned either in an open or percutaneous manner.

The implants are typically positioned such that, pursuant to expansion thereof, and upon application of a force and/or pressure thereto, stress resulting from the force and/or pressure is preferentially distributed toward stronger portions of the vertebral endplates (e.g., peripheral portions of the endplates, such as those portions adjacent to annulus portions of the discs) and away from weaker portions of the vertebral endplates (e.g., interior portions of the endplates, such as those portions adjacent to nucleus portions of the discs). According to other embodiments, the implant may be placed, implanted, or positioned in a joint (e.g., between two adjacent vertebral endplates) such that, pursuant to application of a force to the implant, stress or stresses resulting from the force are modulated and/or distributed in the joint (e.g., between the endplates) in a normalized manner. In a spinal joint that includes a disc, since alterations in the stresses experienced by the annulus of the disc which deviate from what a healthy patient normally experiences is generally acknowledged to be a source of pain, using an implant to normalize and/or modulate stresses upon the annulus in a controlled fashion can alleviate back pain and/or discomfort.

Typically, after the implants have been properly positioned, the implants are expanded and their thickness increases. If several separate and expandable compartments are used to form the cover of an implant, the compartments are typically joined together before their expansion. However, as discussed above, pre-expanded compartments may be used to form the cover.

According to certain embodiments of the present invention, methods of reducing back pain include promoting fibrous in-growth of annulus fibers into a medical implant that is positioned adjacent to a vertebral disc. Such in-growth reduces the ability of the implant to slide or otherwise move once in position and also increases the structural integrity of the patient's spine. When the promotion of such in-growth is desired, the medical implant typically includes biocompatible and/or bioresorbable materials such as, but not limited to, those discussed above.

When positioning an implant according to the methods of reducing back pain that are within the scope of the present invention, at least one bony landmark on at least one of the vertebral bodies and/or endplates that are adjacent to the implant may be referenced. Such referencing helps to ensure proper positioning of the implant. According to certain embodiments of the present invention, the referenced bony landmark is matched to a portion of the cover of the implant that has been manufactured in view of the topography of the disc, as discussed above.

In order to expand a medical implant according to certain embodiments of the present invention, stent technology is used, in some cases as the sole means of expanding the cover and, in some cases, in conjunction with a filler. According to certain embodiments, a compressed or collapsed stent is included either within the cover or on the periphery of the cover. Then, once the implant has been positioned in a patient's body, the stent may be mechanically expanded, thereby expanding the cover. This process may be performed with or without the injection of a liquid, solid, or gas filler into the cover.

Regardless of whether a stent is used or not, the expansion step may restore or increase a patient's intervertebral disc height, thereby reducing or eliminating pain. According to certain embodiments, the implant is expanded incrementally and/or partially. Such an incremental and/or partial expansion of the implant allows for an optimal separation to be obtained between adjacent endplates.

According to certain embodiments of the present invention, methods of reducing back pain include using the implant as a local drug delivery unit that delivers one or more drugs. According to some of these embodiments, one or more drugs (e.g., anti-inflamatories, anti-biotics, local anesthetics, fiber-inducing growth factors, etc.) are included in the implant before the implant is inserted into a patient's body. For example, a coating that includes one or more drugs may be deposited onto the surface of the implant's cover before insertion and the drug(s) may diffuse away from the implant once in the patient's body. According to certain other embodiments, one or more drugs are included as part of a filler material that is inserted into the implant cover once inside the patient's body. When one or more drugs are included in the filler, the drugs are typically able to diffuse out through the cover of the implant.

In addition to matching at least one surface of the medical implant to the topography of structures surrounding the implant, other methods may be used to prevent the implant from moving or sliding once in the body. For example, securing methods such as, but not limited to, sutures, bone anchors, screws, tissue adhesives, and bone adhesives may be used, either alone or in conjunction with each other and/or with a topographically-matched surface to prevent motion of the implant. If the implant is positioned between two vertebral bodies and/or endplates, the securing method(s) may secure the implant to one or more of the disc anulus, the disc nucleus, one or both of the endplate, one or both of the adjacent vertebral bodies, and/or any other rigid structure that is proximate to the implant.

As discussed above, certain embodiments of the present invention specify that an implant be placed between a first vertebral body and a second vertebral body while the implant is in a collapsed state. The collapsed state of the implant allows for the implant to be inserted between the vertebral bodies through a smaller pathway than would be required for a fully-expanded implant. In order to reduce the size of the pathway even further, an implant may be positioned between the vertebral bodies by inserting one compartment of the cover of the implant at a time. Each of these compartments may be expand either before or after being positioned and may be joined together once in position between the vertebral bodies.

A representative method of properly positioning an implant is the percutaneous approach. This approach allows for a smaller pathway than more traditional open approaches because a surgical needle or other needle-type device may be used to insert both the cover and, if necessary, the filler included in the medical implant.

In order to properly place an implant between two vertebral bodies, a number of other approaches may be used. According to certain embodiments of the present invention, a posterior lateral approach is used to insert the implant. According to other embodiments, a posterior approach is used. According to yet other embodiments, an anterior approach is used. However, the above list of approaches is non-limiting of the present invention and those skilled in the art may use other approaches without straying from the scope of the claimed invention.

According to other embodiments of the present invention, an implant is inserted and properly positioned between two vertebral bodies after having traveled through a hole formed in a vertebral body. According to these embodiments, a hole is first formed (e.g., “drilled”) through a vertebral body that is adjacent to the position where the implant is to be located. Then, the implant is inserted and positioned as desired, either as a complete implant or compartment-by-compartment. Finally, the hole is sealed with a material that promotes restoration of the vertebral body to its original condition (e.g., bone cement). This type of approach (i.e., through a hole in a vertebral body) is often preferably used when a patient's spine has been injured or has degenerated in a manner that does not allow for other approaches to be used.

While, in many cases, the above-described implants remain in the patient's body for the remainder of the patient's life, there are situations when the implants may need to be removed and/or replaced. In order to address this need, certain embodiments of the present invention allow for the removal of implants. According to some of these embodiments, an implant that had previously been positioned in a patient's body and expanded is allowed to collapse (e.g., by removing filler from the interior of the cover, mechanically collapsing the stent, etc.). Then, the implant is removed, either in its entirety or compartment by compartment.

While the invention has been described in detail and with reference to specific embodiments thereof, it will be apparent to those skilled in the art that various changes and modifications can be made therein without departing from the spirit and scope thereof. Thus, it is intended that the present invention cover the modifications and variarions of this invention provided they come within the scope of the appended claims and their equivalents.

What is claimed is:

1. A medical implant comprising:
an expandable cover having an interior portion, a peripheral portion that surrounds the interior portion, and appropriate dimensions for insertion between a first vertebral endplate and a second vertebral endplate, wherein, when in an expanded position, the peripheral portion has a thicker cross-section than the interior portion.

2. The implant of claim 1, wherein, when in an expanded position, the cover has a substantially I-shaped cross-section.

3. The implant of claim 1, wherein, when in an expanded position, the cover has a substantially bi-concave cross-section.

4. The implant of claim 1, wherein the cover includes at least one of a hydrophilic material, a bioresorbable material, an inert material, a fluid whose hydrostatic properties may be controlled from a distance, and a biocompatible material.

5. The implant of claim 1, further comprising:
a filler positioned within the expandable cover.

6. The implant of claim 5, wherein the filler includes at least one of a hydrated hydrogel, an non-hydrated hydrogel, a polyvinyl alcohol, an inert gas, an inert fluid, a fluid whose hydrostatic properties may be controlled from a distance, and a biocompatible material.

7. The implant of claim 1, further comprising:
at least two separate compartments formed within the cover.

8. The implant of claim 1, wherein the cover includes a textured surface positioned adjacent to at least one of the first and second vertebral endplate.

9. The implant of claim 8, wherein the textured surface enhances at least one of the implant's lubriciousness and the implant's susceptibility to biological incorporation into a patient's body.

10. The implant of claim 1, wherein the cover includes a surface that is anatomically contoured to bony landmarks found on at least one of the vertebral endplates.

11. A method of manufacturing a medical implant, the method comprising:
forming an expandable cover with an interior portion, a peripheral portion that surrounds the interior portion, and appropriate dimensions for insertion between a first vertebral endplate and a second vertebral endplate, wherein, when in an expanded position, the peripheral portion has a thicker cross-section than the interior portion.

12. The method of claim 11, further comprising:
properly sizing the implant using precise geometric dimensions of at least one of the vertebral endplates.

13. A method of reducing back pain, the method comprising:
placing a medical implant between two adjacent vertebral endplates; and
positioning the implant such that, pursuant to application of a force thereto, stress resulting from the force is preferentially distributed toward stronger portions of the vertebral endplates and away from weaker portions of the vertebral endplates.

14. The method of claim 13, wherein the placing step comprises preferentially distributing the stress to peripheral portions of the vertebral endplates and away from interior portions of the vertebral endplates.

15. The method of claim 13, wherein the placing step comprises placing an expandable medical implant between the vertebral endplates, and further comprising:
expanding the medical implant.

16. The method of claim 15, wherein the expanding step comprises increasing intervertebral disc height.

17. The method of claim 15, wherein the expanding step comprises restoring intervertebral disc height.

18. The method of claim 13, further comprising promoting fibrous in-growth of annulus fibers into the implant.

19. The method of claim 13, wherein the positioning step comprises referencing at least one of bony landmarks on at least one of the endplates to ensure proper positioning of the cover.

20. The method of claim 19, wherein the positioning step comprises using one or more of the bony landmarks to prevent displacement of the cover.

21. The method of claim 14, wherein the expanding step comprises using stent technology.

22. The method of claim 13, further comprising:
locally delivering a drug between the discs via diffusion of the drug out of the implant.

23. The method of claim 13, further comprising:
securing the implant to at least one of the anulus, the nucleus, an endplate, and a vertebral body using at least one of a suture, a bone anchor, a screw, a tissue adhesive, and a bone adhesive.

24. The method of claim 13, wherein the placing step comprises employing at least one of a percutaneous approach, a posterior approach, and an anterior approach for inserting the implant.

25. The method of claim 13, wherein the placing step comprises moving the implant through a hole in a vertebral body.

26. The method of claim 13, wherein the placing step comprises:
inserting a first compartment of the implant and a second compartment of the implant between the two adjacent vertebral endplates; and
joining the first compartment and the second compartment.

27. The method of claim 13, further comprising:
collapsing the implant; and
removing the implant from between the two adjacent vertebral discs.

28. An intervertebral disc implant comprising:
a biocompatible pre-shaped expandable member adapted to fit between two adjacent vertebral bodies, the member having a bottom surface for placement against an uncus and a top surface for placing against an inferior endplate of an adjacent vertebra, wherein said implant contains at least one concavity on said implant's surfaces.

29. A method of reducing back pain, the method comprising:
placing a medical implant between two adjacent vertebral endplates; and
positioning the implant such that, pursuant to application of a force thereto, stress resulting from the force is distributed between the endplates in a normalized manner.

30. The method of claim 29, wherein the positioning step comprises positioning the implant such that the implant modulates the stress upon an annulus in a controlled fashion.
